(19) United States
(12) Patent Application Publication
(10) Pub. No.: US 2026/0090812 A1
(43) Pub. Date: Apr. 2, 2026

(54) TIBIAL ALIGNMENT INSTRUMENT AND METHOD OF USING THE SAME IN AN ORTHOPAEDIC SURGICAL KNEE PROCEDURE

(71) Applicant: DePuy Ireland Unlimited Company, Ringaskiddy (IE)

(72) Inventors: Kyle C. Helm, Fort Wayne, IN (US); Thomas E. Wogoman, Warsaw, IN (US); Daniel E. Lashure, Warsaw, IN (US); Derek A. Shrack, Leesburg, IN (US); George A. Bowman, Bargersville, IN (US); Anees Ahamed Muhamed Farook, Warsaw, IN (US)

(21) Appl. No.: 19/344,898

(22) Filed: Sep. 30, 2025

Related U.S. Application Data
(60) Provisional application No. 63/701,078, filed on Sep. 30, 2024.

(51) Int. Cl. A61B 17/15

(52) U.S. Cl. CPC A61B 17/157

(57) ABSTRACT

An orthopaedic surgical instrument includes an alignment guide having a pair of spacing guides secured thereto. The spacing guides are positioned between an installed femoral trial component and the articular surfaces of a patient's tibia. The alignment guide is secured to the spacing guides so as to align a tibial cutting block on the anterior surface of the patient's tibia based on desired extension gaps. A method of surgically preparing a patient's tibia is also disclosed.

This application claims priority under 35 U.S.C. § 119 to U.S. Provisional Patent Application Ser. No. 63/701,078 which was filed on Sep. 30, 2024 and is incorporated herein by reference in its entirety.

### TECHNICAL FIELD

The present disclosure relates generally to orthopaedic surgical instruments and, more particularly, to surgical instruments used to resect a patient's bone.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

In total knee arthroplasty (TKA), the femur and tibia of the patient's knee are resected to create planar surfaces onto which a prosthetic femoral component and tibial component, respectively, are installed. Traditional TKA involves determining the resection planes based on a pre-determined angle as a function of mechanical alignment or by using a balanced approach that sets the resection planes based on ligament tension. More recently, kinematic alignment techniques involve determining the resection planes as a function of the native, pre-disease state of the patient's knee.

### SUMMARY

According to one aspect of the disclosure, an orthopaedic surgical instrument for use in locating a tibial cutting block during the surgical preparation of a patient's tibia during performance of an orthopaedic knee procedure includes an alignment guide that is selectively securable to, and removable from, the tibial cutting block. The alignment guide has an elongated body extending in the medial/lateral direction. A medial bore is formed in a medial end of the elongated body. The medial bore has a longitudinal axis extending in the anterior/posterior direction. A lateral bore is formed in a lateral end of the elongated body. The lateral bore has a longitudinal axis extending in the anterior/posterior direction so as to be parallel with the longitudinal axis of the medial bore. The alignment guide also has a connector configured to secure the alignment guide to the tibial cutting block. The orthopaedic surgical instrument also includes a medial spacing guide having a medial paddle that includes a superior alignment surface and an opposite inferior surface. The superior alignment surface is sized and shaped to conform to a medial condyle surface of a femoral trial component. The medial spacing guide also includes an elongated shaft extending anteriorly away from the medial paddle. The elongated shaft of the medial spacing guide is positioned in the medial bore of the alignment guide. The orthopaedic surgical instrument also includes a lateral spacing guide having a lateral paddle that includes a superior alignment surface and an opposite inferior surface. The superior alignment surface is sized and shaped to conform to a lateral condyle surface of the femoral trial component. The lateral spacing guide also includes an elongated shaft extending anteriorly away from the lateral paddle. The elongated shaft of the lateral spacing guide is positioned in the lateral bore of the alignment guide.

In an embodiment, the superior alignment surface of the medial spacing guide is sized and shaped to conform to a medial distal condyle surface of the femoral trial component, and the superior alignment surface of the lateral spacing guide is sized and shaped to conform to a lateral distal condyle surface of the femoral trial component.

In an embodiment, the superior alignment surface of the medial spacing guide is sized and shaped to conform to a medial posterior condyle surface of the femoral trial component, and the superior alignment surface of the lateral spacing guide is sized and shaped to conform to a lateral posterior condyle surface of the femoral trial component.

In an embodiment, the inferior alignment surface of the medial spacing guide is configured to be positioned in contact with a medial articular surface of the patient's tibia, and the inferior alignment surface of the lateral spacing guide is configured to be positioned in contact with a lateral articular surface of the patient's tibia.

The connector of the alignment guide may include a tongue secured to the elongated body. The tongue is configured to be received into a cutting guide slot of the tibial cutting block, and is positioned inferiorly of the medial and lateral bores and extends posteriorly away from the elongated body.

In an embodiment, the tongue extends in the anterior/posterior direction such that a superior surface of the tongue is parallel with the longitudinal axes of the medial bore and the lateral bore.

In another embodiment, the tongue extends in the anterior/posterior direction such that a superior surface of the tongue forms a slope angle of 0°-7° with the longitudinal axes of the medial bore and the lateral bore.

According to another aspect, an orthopaedic surgical instrument assembly for use in the surgical preparation a tibia of a patient during performance of an orthopaedic knee procedure includes a femoral trial component configured to be coupled to a surgically-prepared distal end of a femur of the patient. The femoral trial component has an articular side including a medial condyle surface and a lateral condyle surface, and a fixation side that is opposite the articular side and configured to engage the surgically-prepared distal end of the femur of the patient. The surgical instrument assembly also includes a tibial cutting block configured to be coupled to an anterior surface of a proximal end of the tibia of the patient. The tibial cutting block has a cutting guide slot that extends in the anterior/posterior direction. The surgical instrument assembly also includes an alignment guide that is selectively securable to, and removable from, the tibial cutting block. The alignment guide has an elongated body extending in the medial/lateral direction, and a connector configured to secure the alignment guide to the tibial cutting block. The surgical instrument assembly further includes a medial spacing guide having a medial paddle that includes a superior alignment surface and an opposite inferior surface. The superior alignment surface is sized and shaped to conform to the medial condyle surface of the femoral trial component. The medial spacing guide also includes an elongated shaft extending anteriorly away from the medial paddle. The elongated shaft of the medial spacing guide is secured to the alignment guide. Yet further, the surgical instrument assembly includes a lateral spacing guide having a lateral paddle that includes a superior alignment surface and an opposite inferior surface. The superior alignment surface is sized and shaped to conform to the lateral condyle surface of the femoral trial component. The lateral spacing guide also includes an elongated shaft extending anteriorly away from the lateral paddle. The elongated shaft of the medial spacing guide is secured to the alignment guide.

In an embodiment, the superior alignment surface of the medial spacing guide is sized and shaped to conform to a medial distal condyle surface of the femoral trial component, and the superior alignment surface of the lateral spacing guide is sized and shaped to conform to a lateral distal condyle surface of the femoral trial component.

In an embodiment, the superior alignment surface of the medial spacing guide is sized and shaped to conform to a medial posterior condyle surface of the femoral trial component, and the superior alignment surface of the lateral spacing guide is sized and shaped to conform to a lateral posterior condyle surface of the femoral trial component.

In an embodiment, the inferior alignment surface of the medial spacing guide is configured to be positioned in contact with a medial articular surface of the patient's tibia, and the inferior alignment surface of the lateral spacing guide is configured to be positioned in contact with a lateral articular surface of the patient's tibia.

The connector of the alignment guide may include a tongue secured to the elongated body. The tongue is configured to be received into a cutting guide slot of the tibial cutting block, and is positioned inferiorly of the medial and lateral bores and extends posteriorly away from the elongated body.

In an embodiment, the elongated shaft of the medial spacing guide has a longitudinal axis that extends in the anterior/posterior direction, with the elongated shaft of the lateral spacing guide having a longitudinal axis that extends in the anterior/posterior direction so as to be parallel with the longitudinal axis of the elongated shaft of the medial spacing guide. The tongue extends in the anterior/posterior direction such that a superior surface of the tongue is parallel with the longitudinal axes of the elongated shafts of the medial spacing guide and the lateral spacing guide.

In an embodiment, the elongated shaft of the medial spacing guide has a longitudinal axis that extends in the anterior/posterior direction, with the elongated shaft of the lateral spacing guide having a longitudinal axis that extends in the anterior/posterior direction so as to be parallel with the longitudinal axis of the elongated shaft of the medial spacing guide. The tongue extends in the anterior/posterior direction such that a superior surface of the tongue forms a slope angle of 0°-7° with the longitudinal axes of the elongated shafts of the medial spacing guide and the lateral spacing guide.

According to another aspect, a method of surgically preparing a tibia of a patient during performance of an orthopaedic knee procedure, includes installing a femoral trial component on a surgically-prepared distal end of a femur of the patient. The method also includes determining an amount of cartilage loss on an articular surface of the tibia of the patient. A spacing guide is selected that includes a paddle having a thickness that corresponds to the determined amount of cartilage loss on the articular surface of the tibia of the patient from a plurality of spacing guides each of which has a paddle of differing thicknesses. The paddle of the selected spacing guide is installed between a condyle surface of the installed femoral trial component and the articular surface of the tibia of the patient. The method includes securing a tibial cutting block to an alignment guide. The alignment guide is secured to the installed spacing guide such that a backside surface of the tibial cutting block abuts an anterior surface of the tibia of the patient.

The method also includes pinning the tibial cutting block to the anterior surface of the tibia of the patient, and thereafter performing a planar resection on the tibia of the patient by use of the pinned tibial cutting block.

In an embodiment, an amount of cartilage loss on a medial articular surface of the tibia of the patient is determined and a spacing guide that includes a paddle having a thickness that corresponds to the determined amount of cartilage loss on the medial articular surface of the tibia of the patient is selected from a plurality of spacing guides each of which has a paddle of differing thicknesses. The paddle of the selected spacing guide is installed between a medial condyle surface of the installed femoral trial component and the medial articular surface of the tibia of the patient.

In an embodiment, an amount of cartilage loss on a lateral articular surface of the tibia of the patient is determined and a spacing guide that includes a paddle having a thickness that corresponds to the determined amount of cartilage loss on the lateral articular surface of the tibia of the patient is selected from a plurality of spacing guides each of which has a paddle of differing thicknesses. The paddle of the selected spacing guide is installed between a lateral condyle surface of the installed femoral trial component and the lateral articular surface of the tibia of the patient.

In another embodiment, an amount of cartilage loss on both a medial articular surface of the tibia of the patient and a lateral articular surface of the tibia of the patient is determined. A medial spacing guide that includes a paddle having a thickness that corresponds to the determined amount of cartilage loss on the medial articular surface of the tibia of the patient is selected from a plurality of spacing guides each of which has a paddle of differing thicknesses. A lateral spacing guide that includes a paddle having a thickness that corresponds to the determined amount of cartilage loss on the lateral articular surface of the tibia of the patient is selected from a plurality of spacing guides each of which has a paddle of differing thicknesses. The paddle of the selected medial spacing guide is installed between a medial condyle surface of the installed femoral trial component and the medial articular surface of the tibia of the patient, and the paddle of the selected lateral spacing guide is installed between a lateral condyle surface of the installed femoral trial component and the lateral articular surface of the tibia of the patient.

A depth probe may be advanced into cartilage of the articular surface of the tibia of the patient to determine cartilage loss.

According to another aspect, a method of surgically preparing a tibia of a knee of a patient during performance of an orthopaedic knee procedure includes installing a femoral trial component on a surgically-prepared distal end of a femur of the patient. The method also includes determining, with the patient's knee positioned in extension, (i) a desired medial extension gap indicative of a distance between a medial condyle of the patient's femur and a medial plateau of the patient's tibia, and (ii) a desired lateral extension gap indicative of a distance between a lateral condyle of the patient's femur and a lateral plateau of the patient's tibia. The method also includes selecting a medial spacing guide that includes a paddle having a thickness that corresponds to the determined medial extension gap from a plurality of spacing guides each of which has a paddle of differing thicknesses, and selecting a lateral spacing guide that includes a paddle having a thickness that corresponds to the determined lateral extension gap from the plurality of spacing guides each of which has a paddle of differing thicknesses. The method also includes installing (i) the paddle of the selected medial spacing guide between a medial condyle surface of the installed femoral trial component and the medial plateau of the tibia of the patient, and (ii) installing the paddle of the selected lateral spacing guide between a lateral condyle surface of the installed femoral trial component and the lateral tibial plateau of the tibia of the patient. The method further includes securing a tibial cutting block to an alignment guide, and securing the alignment guide to the installed spacing guide such that a backside surface of the tibial cutting block abuts an anterior surface of the tibia of the patient.

The desired medial extension gap and the desired lateral extension gap may be determined by operating a ligament balancer to balance the patient's knee.

The desired medial extension gap and the desired lateral extension gap may be determined by determining an amount of cartilage loss within the patient's knee.

The selected medial and lateral spacing guides may be installed with the patient's knee positioned in extension.

The selected medial and lateral spacing guides may be installed with the patient's knee positioned in flexion.

According to another aspect, an orthopaedic surgical instrument assembly for use in the surgical preparation a tibia of a patient during performance of an orthopaedic knee procedure includes a femoral trial component configured to be coupled to a surgically-prepared distal end of a femur of the patient. The femoral trial component has an articular side that includes a medial condyle surface and a lateral condyle surface, and a fixation side that is opposite the articular side and configured to engage the surgically-prepared distal end of the femur of the patient. The femoral trial component also includes a medial lug hole positioned at a dwell point of the medial condyle surface and extending from the articular side to the fixation side, and a lateral lug hole positioned at a dwell point of the lateral condyle surface and extending from the articular side to the fixation side. The surgical instrument assembly also includes a medial spacing guide having a medial paddle that includes a superior alignment surface and an opposite inferior surface. The superior alignment surface is sized and shaped to conform to the medial condyle surface of the femoral trial component. The medial spacing guide also includes an elongated shaft extending anteriorly away from the medial paddle, the elongated shaft of the medial spacing guide being secured to the alignment guide. The medial alignment guide also includes an upwardly extending post formed in the superior alignment surface of the medial spacing guide. The upwardly extending post of the medial spacing guide is sized and shaped to be friction fit into the medial lug hole of the femoral trial component. The surgical instrument assembly also includes a lateral spacing guide having a lateral paddle that includes a superior alignment surface and an opposite inferior surface. The superior alignment surface is sized and shaped to conform to the lateral condyle surface of the femoral trial component. The lateral spacing guide also includes an elongated shaft extending anteriorly away from the lateral paddle. The elongated shaft of the medial spacing guide is secured to the alignment guide. The lateral spacing guide also includes an upwardly extending post formed in the superior alignment surface of the lateral spacing guide. The upwardly extending post of the lateral spacing guide is sized and shaped to be friction fit into the lateral lug hole of the femoral trial component.

In an embodiment, the superior alignment surface of the medial spacing guide is sized and shaped to conform to a medial distal condyle surface of the femoral trial component, and the superior alignment surface of the lateral spacing guide is sized and shaped to conform to a lateral distal condyle surface of the femoral trial component.

In an embodiment, the superior alignment surface of the medial spacing guide is sized and shaped to conform to a medial posterior condyle surface of the femoral trial component, and the superior alignment surface of the lateral spacing guide is sized and shaped to conform to a lateral posterior condyle surface of the femoral trial component.

In an embodiment, the inferior alignment surface of the medial spacing guide is configured to be positioned in contact with a medial articular surface of the patient's tibia, and the inferior alignment surface of the lateral spacing guide is configured to be positioned in contact with a lateral articular surface of the patient's tibia.

In an embodiment, the elongated shaft of the medial spacing guide has a longitudinal axis that extends in the anterior/posterior direction, with the elongated shaft of the lateral spacing guide having a longitudinal axis that extends in the anterior/posterior direction so as to be parallel with the longitudinal axis of the elongated shaft of the medial spacing guide.

According to another aspect, a method of surgically preparing a tibia of a patient during performance of an orthopaedic knee procedure, includes installing a femoral trial component on a surgically-prepared distal end of a femur of the patient. The method also includes determining an amount of cartilage loss on an articular surface of the tibia of the patient. A spacing guide is selected that includes a paddle having a thickness that corresponds to the determined amount of cartilage loss on the articular surface of the tibia of the patient from a plurality of spacing guides each of which has a paddle of differing thicknesses. The method also includes inserting a post of the paddle of the selected spacing guide into a lug hole formed at a dwell point of a condyle surface of the installed femoral trial component so as to install the paddle of the selected spacing guide between the condyle surface of the installed femoral trial component and the articular surface of the tibia of the patient.

In an embodiment, an amount of cartilage loss on a medial articular surface of the tibia of the patient is determined and a spacing guide that includes a paddle having a thickness that corresponds to the determined amount of cartilage loss on the medial articular surface of the tibia of the patient is selected from a plurality of spacing guides each of which has a paddle of differing thicknesses. The post of the selected spacing guide is inserted into a lug hole formed in the medial condyle surface of the femoral trial component. The paddle of the selected spacing guide is installed between a medial condyle surface of the installed femoral trial component and the medial articular surface of the tibia of the patient.

In an embodiment, an amount of cartilage loss on a lateral articular surface of the tibia of the patient is determined and a spacing guide that includes a paddle having a thickness that corresponds to the determined amount of cartilage loss on the lateral articular surface of the tibia of the patient is selected from a plurality of spacing guides each of which has a paddle of differing thicknesses. The post of the selected spacing guide is inserted into a lug hole formed in the lateral condyle surface of the femoral trial component. The paddle of the selected spacing guide is installed between a lateral condyle surface of the installed femoral trial component and the lateral articular surface of the tibia of the patient.

In another embodiment, an amount of cartilage loss on both a medial articular surface of the tibia of the patient and a lateral articular surface of the tibia of the patient is determined. A medial spacing guide that includes a paddle having a thickness that corresponds to the determined amount of cartilage loss on the medial articular surface of the tibia of the patient is selected from a plurality of spacing guides each of which has a paddle of differing thicknesses. A lateral spacing guide that includes a paddle having a thickness that corresponds to the determined amount of cartilage loss on the lateral articular surface of the tibia of the patient is selected from a plurality of spacing guides each of which has a paddle of differing thicknesses. The post of the selected medial spacing guide is inserted into a lug hole formed in the medial condyle surface of the femoral trial component, and the post of the selected lateral spacing guide is inserted into a lug hole formed in the lateral condyle surface of the femoral trial component. The paddle of the selected medial spacing guide is installed between a medial condyle surface of the installed femoral trial component and the medial articular surface of the tibia of the patient, and the paddle of the selected lateral spacing guide is installed between a lateral condyle surface of the installed femoral trial component and the lateral articular surface of the tibia of the patient.

A depth probe may be advanced into cartilage of the articular surface of the tibia of the patient to determine cartilage loss.

According to another aspect, a method of surgically preparing a tibia of a knee of a patient during performance of an orthopaedic knee procedure includes installing a femoral trial component on a surgically-prepared distal end of a femur of the patient. The method also includes determining, with the patient's knee positioned in extension, (i) a desired medial extension gap indicative of a distance between a medial condyle of the patient's femur and a medial plateau of the patient's tibia, and (ii) a desired lateral extension gap indicative of a distance between a lateral condyle of the patient's femur and a lateral plateau of the patient's tibia. The method also includes selecting a medial spacing guide that includes a paddle having a thickness that corresponds to the determined medial extension gap from a plurality of spacing guides each of which has a paddle of differing thicknesses, and selecting a lateral spacing guide that includes a paddle having a thickness that corresponds to the determined lateral extension gap from the plurality of spacing guides each of which has a paddle of differing thicknesses. The method further includes inserting (i) a post of the paddle of the selected medial spacing guide into a lug hole formed at a dwell point of a medial condyle surface of the installed femoral trial component, and (ii) a post of the paddle of the selected lateral spacing guide into a lug hole formed at a dwell point of a lateral condyle surface of the installed femoral trial component. The method also includes installing (i) the paddle of the selected medial spacing guide between a medial condyle surface of the installed femoral trial component and the medial plateau of the tibia of the patient, and (ii) the paddle of the selected lateral spacing guide between a lateral condyle surface of the installed femoral trial component and the lateral tibial plateau of the tibia of the patient.

The desired medial extension gap and the desired lateral extension gap may be determined by operating a ligament balancer to balance the patient's knee.

The desired medial extension gap and the desired lateral extension gap may be determined by determining an amount of cartilage loss within the patient's knee.

The selected medial and lateral spacing guides may be installed with the patient's knee positioned in extension.

The selected medial and lateral spacing guides may be installed with the patient's knee positioned in flexion.

### BRIEF DESCRIPTION OF THE DRAWINGS

The detailed description particularly refers to the following figures, in which:

FIG. 1 is a perspective view of a tibial alignment instrument for use in the surgical preparation of a patient's tibia during performance of an orthopaedic knee procedure, note the tibial cutting block is secured to the tibial alignment instrument in FIG. 1;

FIG. 2 is side view of the tibial alignment instrument of FIG. 1, note that, similarly to FIG. 1, the tibial cutting block is secured to the tibial alignment instrument in FIG. 2;

FIG. 3 is a perspective view showing the spacing guide of the tibial alignment instrument in various differing sizes;

FIG. 4 is a fragmentary side elevation view showing the various versions of the alignment guide of the tibial alignment instrument each of which having a tongue with a differing angle;

FIG. 5 is a view similar to FIG. 2, but showing the tibial cutting block being positioned at various differing angles based on the angle of the tongue of the alignment guide of the tibial alignment instrument;

FIG. 6 is a side view showing the tibial alignment instrument installed in the knee joint of the patient prior to performance of a proximal resection of the patient's tibia, note the patient's knee is shown in extension in FIG. 6;

FIG. 7 is a frontal view showing the tibial alignment instrument installed in the knee joint of the patient prior to performance of a proximal resection of the patient's tibia, note the patient's knee is shown in extension in FIG. 7;

FIG. 8 is a side view showing the tibial cutting block installed on the anterior surface of the patient's tibia and being used during performance of a proximal resection of the patient's tibia;

FIG. 9 is a side view similar to FIG. 6, but showing the tibial alignment instrument installed in the knee joint of the patient with the patient's knee positioned in flexion; and

FIG. 10 is a side view similar to FIG. 6, but showing another embodiment of the spacing guides of the tibial alignment instrument.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

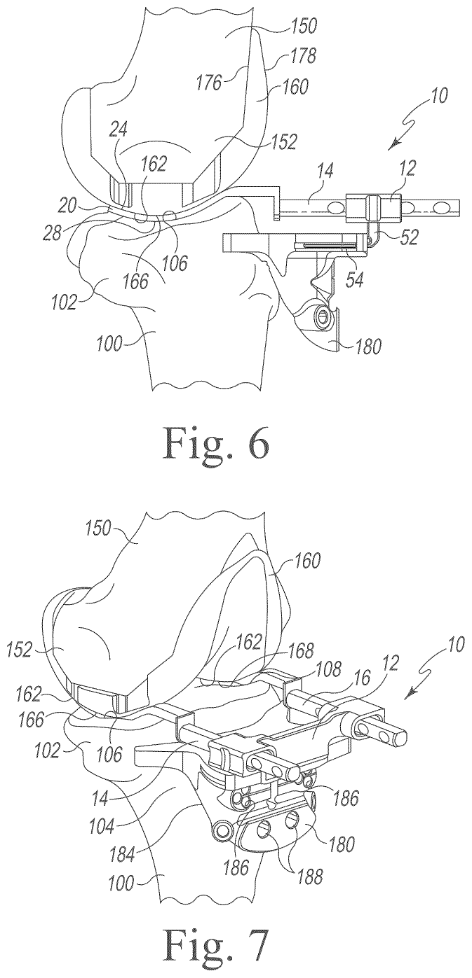

Referring to FIGS. 1-5, an orthopaedic surgical instrument 10—the form of a tibial alignment instrument-for use in the surgical preparation of a patient's tibia during performance of an orthopaedic knee procedure is shown. The tibial alignment instrument 10 includes an alignment guide 12 and a pair of removable spacing guides 14, 16. The tibial alignment instrument 10 is used to locate a tibial cutting block 180 during the surgical preparation of a patient's tibia during performance of an orthopaedic knee procedure (see FIGS. 6 and 7). As will be discussed below in greater detail, once so located and installed with guide pins, the tibial cutting block 180 is used to perform a proximal cut on the patient's tibia (see FIG. 9).

As alluded to above and shown in FIGS. 1-5, the tibial alignment instrument 10 includes a pair of spacing guides 14, 16 configured to guide the placement of the tibial cutting block 180 on the anterior surface 104 of the proximal end 102 of a patient's tibia 100 (see FIGS. 6 and 7). Each of the spacing guides 14, 16 includes a paddle 20, 22, respectively. The paddles 20, 22 include superior alignment surfaces 24, 26, respectively, and opposite inferior surfaces 28, 30, respectively. The superior alignment surfaces 24, 26 and the inferior surfaces 28, 30 cooperate with the condyle surfaces of a femoral trial component 160 secured to a surgically-prepared distal end 152 of the patient's femur 150 and the articular surfaces of the patient's tibia 100. Specifically, as will be discussed below in more detail, the alignment surface 24 of the medial paddle 20 is sized and shaped to conform to a medial condyle surface 162 of the femoral trial component 160, whereas the alignment surface 26 of the lateral paddle 22 is sized and shaped to conform to a lateral condyle 164 of the femoral trial component 160. As shown in FIGS. 6 and 7, the inferior surface 28 of the medial paddle 20 is configured to be positioned in contact with a medial articular surface 106 of the patient's tibia 100, whereas the inferior surface 30 of the lateral paddle 22 is configured to be positioned in contact with a lateral articular surface 108 of the patient's tibia 100. As shown in FIGS. 6 and 7 and discussed below in greater detail, such an arrangement of the paddles 20, 22 allows them to create a gap of a desired distance between the medial condyle 162 of femoral trial component 160 and the medial articular surface 106 of the patient's tibia 100 and a gap of a desired distance between the lateral condyle 164 of femoral trial component 160 and the lateral articular surface 108 of the patient's tibia 100, respectively.

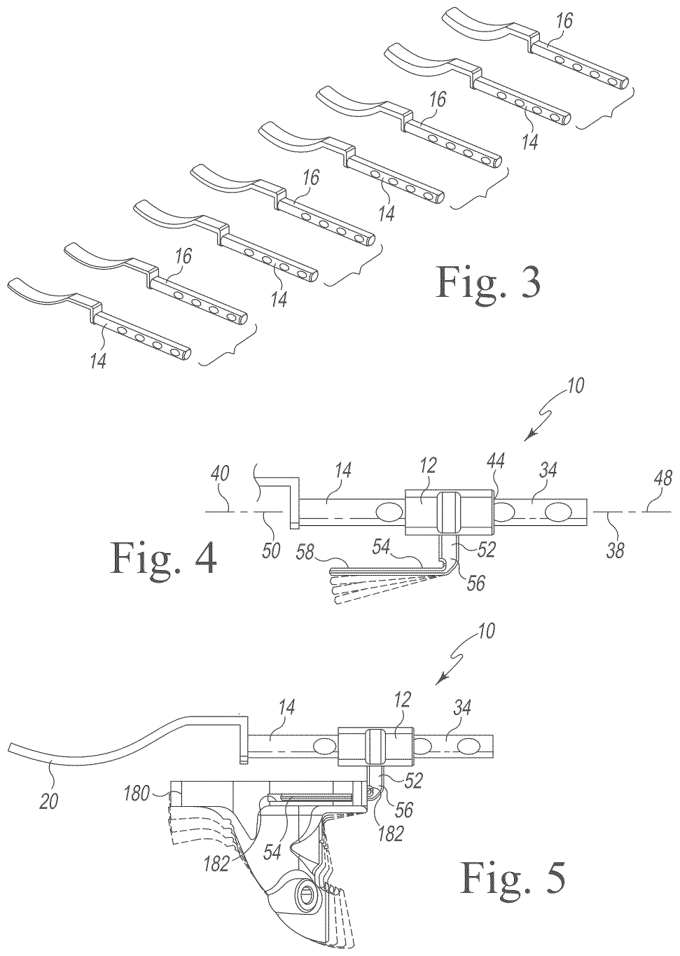

To this end, as shown in FIG. 3, the paddles 20, 22 of the spacing guides 14, 16 are configured with varying different sizes (i.e., different thicknesses) so that the surgeon can intraoperatively select a best-fit option to pair with the alignment guide 12. For example, the paddles 20, 22 of the spacing guides 14, 16 may be provided in size 1 (e.g., having a 1 mm thickness), size 2 (e.g., having a 2 mm thickness), size 3 (e.g., having a 3 mm thickness), and size 4 (e.g., having a 4 mm thickness). The paddles 20, 22 may also be provided in additional sizes to fit the needs of a given design of the tibial alignment instrument 10. Moreover, depending on the curvature of the condyle surfaces of a given femoral trial component 160, the spacing guides 14, 16 may be unique to each side of the component 160—that is, a specific spacing guide may be used with the medial side of the femoral trial component 160, but not the lateral side and vice versa. Alternatively, in some embodiments, the spacing guides 14, 16 may be “universal” and, thus, not unique to either side of the femoral trial component 160—that is, a specific spacing guide may be used with both the medial side and the lateral side of the femoral trial component 160.

Further, the superior alignment surfaces 24, 26 of the paddles 20, 22 of the spacing guides 14, 16 may be configured based on whether the tibial alignment instrument 10 is used with the patient's knee positioned in extension (see FIGS. 6 and 7) or positioned in flexion (see FIG. 9). In particular, if the tibial alignment instrument 10 is used with the patient's knee positioned in extension (see FIGS. 6 and 7), the superior alignment surfaces 24, 26 of the paddles 20, 22 of the spacing guides 14, 16 may be configured (i.e., sized and shaped) to conform to the medial distal condyle surface 166 of the femoral trial component 160 and the lateral distal condyle surface 168 of the femoral trial component 160, respectively. However, if the tibial alignment instrument 10 is used with the patient's knee positioned in flexion (see FIG. 9), the superior alignment surfaces 24, 26 of the paddles 20, 22 of the spacing guides 14, 16 may be configured (i.e., sized and shaped) to conform to the medial posterior condyle surface 170 of the femoral trial component 160 and the lateral posterior condyle surface 172 of the femoral trial component 160, respectively. It should be appreciated that in some embodiments in which the curvatures of the distal condyle surfaces 166, 168 and the posterior condyle surfaces 170, 172 of the femoral trial component 160 are similar or nearly similar to one another, the superior alignment surfaces 24, 26 of the paddles 20, 22 of the spacing guides 14, 16 may be “universal” and, thus, not unique to the position of the patient's knee—that is, a given spacing guide 14, 16 may be used irrespective of whether it's being used with the patient's knee positioned in extension or positioned in flexion.

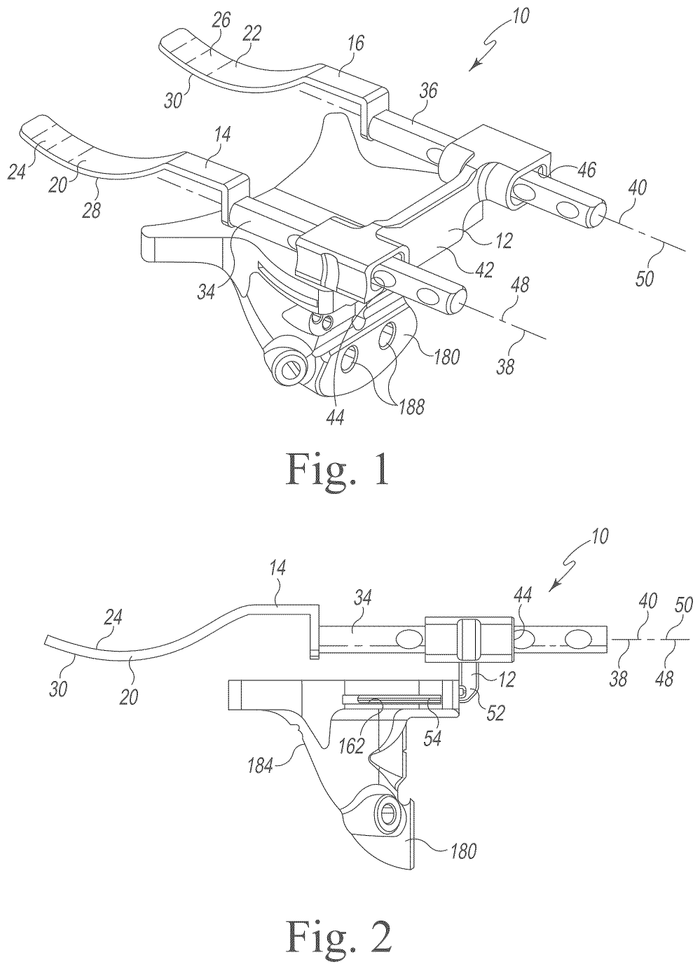

As can be seen in FIGS. 1-5, the spacing guides 14, 16 each include an elongated shaft 34, 36, respectively, that extends anteriorly away from the guide's respective paddle 20, 22. As will be described below in greater detail, the alignment guide 12 is secured to the shafts 34, 36 during use of the tibial alignment instrument 10. As can be seen in FIGS. 1 and 2, the respective longitudinal axes 38, 40 of the elongated shafts 34, 36 extend in the anterior/posterior direction and are parallel with one another. Moreover, as can be seen best in FIGS. 3-5, the elongated shafts 34, 36 of the spacing guides 14, 16 have an indented or similarly textured outer surface. Such a textured surface increases the surgeon's ability to grip the spacing guides 14, 16 during installation thereof, particularly in the presence of the fluids commonly present during a surgical procedure.

As can be seen in FIGS. 1-5, the alignment guide 12 is used to couple the tibial cutting block 180 to the spacing guides 14, 16. The alignment guide 12 has an elongated body 42 that extends in the medial/lateral direction between the spacing guides 14, 16. The ends of the elongated body 42 have bores 44, 46 formed therein into which the elongated shafts 34, 36 of the spacing guides 14, 16 are respectively received. In particular, the medial bore 44 is formed in the medial end of the elongated body 42, whereas the lateral bore 46 is formed in the lateral end of the elongated body 42. As can be seen in FIGS. 1 and 2, the respective longitudinal axes 48, 50 of the bores 44, 46 extend in the anterior/posterior direction and are parallel with one another. As such, when the alignment guide 12 is installed on the spacing guides 14, 16, the longitudinal axes 38, 40 of the elongated shafts 34, 36 are coaxial with the longitudinal axes 48, 50 of the bores 44, 46, respectively. In other words, the elongated shaft 34 of the medial spacing guide 14 and the medial bore 44 of the alignment guide 12 lie on a common longitudinal axis 38, 48. Similarly, the elongated shaft 36 of the lateral spacing guide 16 and the lateral bore 46 of the alignment guide 12 lie on a common longitudinal axis 40, 50.

The alignment guide 12 also includes a connector 52 configured to secure the alignment guide 12 to the tibial cutting block 180 such that the guide 12 may be selectively securable to, and removable from, the block 12. As can be best seen in FIGS. 4 and 5, the connector 52 includes a tongue 54 secured to the alignment guide's elongated body 42. The tongue 54 is configured to be received into a cutting guide slot 182 of the tibial cutting block 180 so as to secure the alignment guide 12 to the block 180. The tongue 54 is positioned inferiorly of the elongated body's medial and lateral bores 44, 46 and extends posteriorly away from the elongated body 42. In particular, a connecting flange 56 extends inferiorly from the elongated body 42, and the tongue 54 is cantilevered to the connecting flange 56. Specifically, with its anterior end secured to the connecting flange 56, the tongue 54 extends posteriorly away from the connecting flange 56 to its free posterior end. By sliding the free posterior end of the tongue 54 into the cutting guide slot 182 of the tibial cutting block 180, the block 180 may be selectively coupled to the alignment guide 12 and hence the tibial alignment instrument 10.

As shown in solid lines in FIGS. 4 and 5, the tongue 54 extends in the anterior/posterior direction such that a superior surface 58 of the tongue 54 is parallel with the longitudinal axes 48, 50 of the medial bore 44 and the lateral bore 46 of the alignment guide 12 (and hence also parallel to the longitudinal axes 38, 40 of the elongated shafts 34, 36 positioned in the bores 44, 46). Such an arrangement positions the cutting guide slot 182 of the tibial cutting block 180 to produce a planar tibial cut on the proximal tibia 102 with no anterior/posterior slope (i.e., an A/P slope angle of zero degrees). However, in some surgical cases, the surgeon may desire to install a tibial tray (not shown) at a small A/P slope angle, typically between three and seven degrees. In order to do so, the planar tibial cut on the proximal tibia is cut at the desired slope angle. To accommodate such cases, the alignment guide may be provided in different versions each of which having its tongue 54 arranged an angle relative to the bores 44, 46 of the alignment guide 12 (and hence also the elongated shafts 34, 36 positioned in the bores 44, 46). Specifically, as shown in phantom lines in FIGS. 4 and 5, in each of such differing versions of the alignment guide 12, the tongue 54 extends in the anterior/posterior direction such that the superior surface 58 of the tongue 54 forms a slope angle of three to seven degrees with the longitudinal axes 48, 50 of the medial bore 44 and the lateral bore 46 of the alignment guide 12 (and hence also parallel to the longitudinal axes 38, 40 of the elongated shafts 34, 36 positioned in the bores 44, 46). Such an arrangement positions the cutting guide slot 182 of the tibial cutting block 180 to produce a planar tibial cut on the proximal tibia 102 with a corresponding anterior/posterior slope angle (i.e., an A/P slope angle of three to seven degrees). In an illustrative embodiment, a kit of four alignment guides 12 may be provided. In such a kit, separate alignment guides having slope angles of 0°, 3°, 5°, and 7° are included.

It should be appreciated that in other embodiments the connector 52 may take the form of elongated pins or shafts, which are received in the instrument guide holes 188 of the cutting block 180 (see FIGS. 1 and 7). Like the tongue 54, such elongated pins extend in the anterior/posterior direction such that a longitudinal axis of each pin extends parallel with the longitudinal axes 48, 50 of the medial bore 44 and the lateral bore 46 of the alignment guide 12 (and hence also parallel to the longitudinal axes 38, 40 of the elongated shafts 34, 36 positioned in the bores 44, 46). Some versions of such alignment guides may have their elongated pins of the connector 52 arranged an angle relative to the bores 44, 46 of the alignment guide 12.

In operation, the surgeon may utilize the tibial alignment instrument 10 during performance of an orthopaedic knee procedure to prepare the proximal end 102 of a patient's tibia 100 to receive a prosthetic tibial tray component. To do so, the surgeon may utilize the tibial alignment instrument 10 to install the tibial cutting block 180 on the anterior surface 104 of the proximal end 102 of a patient's tibia 100 and thereafter use the installed cutting block 180 to guide a bone saw blade 190 in making a proximal cut on the proximal end 102 of a patient's tibia 100.

During such an orthopaedic surgical procedure, the surgeon first surgically prepares the distal end 152 of the patient's femur 150 and thereafter installs the femoral trial component 160. In doing so, the surgeon engages the fixation side 176 of the femoral trial component 160 with the surgically-prepared distal end 152 of the femur 150 such that its opposite articular side 178 (i.e., the side including the condyle surfaces 162, 164) faces the articular surfaces 106, 108 of the patient's tibia 100. The surgeon then orientates the patient's femur 150 and tibia 100 such that the patient's knee is positioned in extension. With the patient's knee positioned in extension, the surgeon may use any of numerous known techniques to determine the desired extension gaps on each side (i.e., the medial and lateral sides) of the patient's knee. In particular, the surgeon may use any of numerous known techniques to determine a desired medial extension gap indicative of the distance between the medial distal condyle surface 166 of the femoral trial component 160 and the articular surface 106 on the medial plateau of the patient's tibia 100. The surgeon may also use any of such known techniques to determine a desired lateral extension gap indicative of the distance between the lateral distal condyle surface 168 of the femoral trial component 160 and the articular surface 108 of the lateral plateau of the patient's tibia 100.

The specific technique used by the surgeon to determine the desired medial extension gap and the desired lateral extension gap (with the patient's knee positioned in extension) may be selected based on the needs of a given surgical procedure or the preferences of a given surgeon. Amongst other techniques, the surgeon may use a patient-specific/kinematic alignment technique in which the surgeon measures cartilage loss and/or other tissue loss within the patient's knee to position the patient's knee in a position that represents the native, pre-disease state of the patient's knee and thereafter determines the desired gap on each side based on the measured cartilage loss. The surgeon may employ numerous techniques for measuring cartilage loss on the articular surfaces 106, 108 of the patient's tibia 100. In one technique, the surgeon determines the amount of cartilage loss on an affected area of the articular surface 106 on the medial plateau of the patient's tibia 100 (i.e., an area of the articular surface 106 exhibiting cartilage loss) by inserting a graduated depth probe (not shown) or similar instrument into the affected area of the articular surface 106 and then also inserting the graduated depth probe into an unaffected area of the articular surface 106 (i.e., an area of the articular surface 106 that does not exhibit cartilage loss). The difference between the two depth measurements reflects the amount of cartilage loss on the articular surface 106 on the medial plateau of the patient's tibia 100. The surgeon then repeats the process on the articular surface 108 on the lateral plateau of the patient's tibia 100. Specifically, the surgeon inserts the graduated depth probe into both the affected area of the lateral articular surface 108 and an unaffected area of the lateral articular surface 108 with the difference between the two depth measurements reflecting the amount of cartilage loss on the lateral articular surface 108.

As a further example, the surgeon may use a balanced approach in which the surgeon operates a ligament balancer to position the femur 150 and the tibia 100 of the patient's knee based in ligament tension and then measures (i) the distance between the medial distal condyle surface 166 of the femoral trial component 160 and the articular surface 106 on the medial plateau of the patient's tibia 100, and (ii) the distance between the lateral distal condyle surface 168 of the femoral trial component 160 and the articular surface 108 on the lateral plateau of the patient's tibia 100.

It should be appreciated that any of such techniques may be performed using mechanical instruments, automated instruments, computer-assisted surgical systems, or any other type of instrument, system, or method conventionally used by surgeons to determine the desired extension gaps with the patient's knee positioned in extension.

Based on the desired extension gaps (as previously determined by the surgeon with the patient's knee positioned in extension), the surgeon then installs the spacing guides 14, 16 into the patient's knee. Specifically, the surgeon selects spacing guides 14, 16 having appropriately sized paddles 20, 22 based on the previously-determined desired medial and lateral extension gaps, respectively. In particular, the surgeon selects a spacing guide 14, 16 with a paddle 20, 22 having a size (i.e., thickness) which corresponds to the desired extension gaps on each side of the patient's knee (i.e., the medial and lateral sides) and installs it between the respective distal condyle surfaces 166, 168 of the femoral trial component 160 and the articular surfaces 106, 108 of the patient's tibia 100. For example, if the surgeon previously determined that the medial side of the patient's knee has a desired extension gap of 4 mm, the surgeon selects a medial spacing guide 14 with a size 4 (4 mm thick) paddle 20 and installs it between the medial distal condyle surface 166 of the femoral trial component 160 and the medial articular surface 106 of the patient's tibia 100. Similarly, for example, if the surgeon previously determined that the lateral side of the patient's knee has a desired extension gap of 2 mm, the surgeon selects a lateral spacing guide 16 with a size 2 (2 mm thick) paddle 22 and installs it between the lateral distal condyle surface 168 of the femoral trial component 160 and the lateral articular surface 108 of the patient's tibia 100.

Once the surgeon has installed both spacing guides 14, 16, the surgeon positions a tibial cutting block 180 of the appropriate size on the alignment guide 12 by sliding the guide's tongue 54 into the anterior opening of the cutting guide slot 182 of the tibial cutting block 180 so as to couple the guide 12 to the block 180. The surgeon then positions the assembled instruments in the patient's knee by advancing the alignment guide 12 such that the elongated shafts 34, 36 of the spacing guides 14, 16 are received into the respective bores 44, 46 of the alignment guide 12. The surgeon then slides the alignment guide 12 posteriorly along the elongated shafts 34, 36 until a backside surface 184 of the tibial cutting block 182 abuts the anterior surface 104 of the patient's tibia 100, as shown in FIGS. 6 and 7. Doing so positions the tibial cutting block 180 in a position based off the desired extension gaps (as they were previously determined) since the size (i.e., thickness) of the installed paddles 20, 22 account for the desired extension gaps.

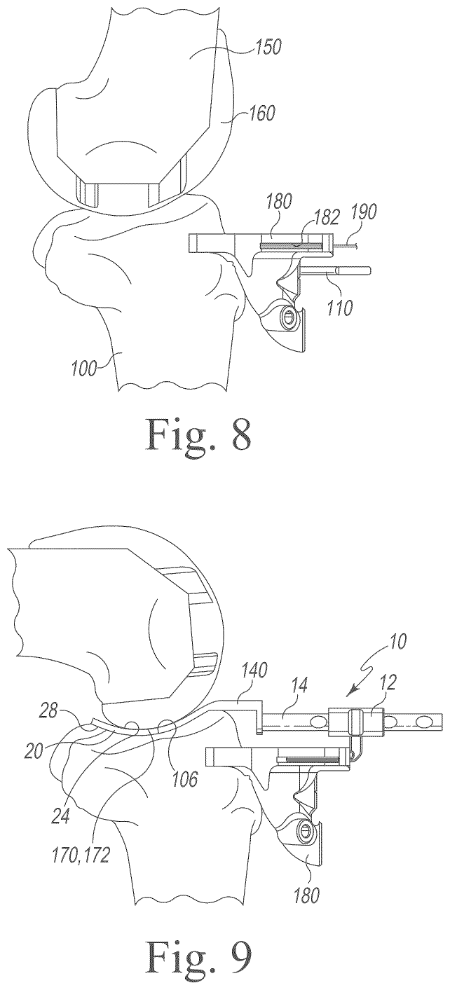

Once the surgeon is satisfied with the alignment of the tibial cutting block 180, the surgeon may pin the tibial cutting block 180 to the patient's tibia 180 by installing a pair of guide pins 110 through a selected pair of the block's pin guide bores 186. With the tibial cutting block 180 pinned to the proximal end 102 of the patient's tibia 100, the surgeon removes the alignment guide 12 from the pinned cutting block 180 and thereafter removes both spacing guides 14, 16, as shown in FIG. 8. As also shown in FIG. 8, the surgeon may then use the tibial cutting block 180 to perform a proximal resection of the proximal end 102 of the patient's tibia 100. Specifically, the surgeon may advance a bone saw blade 112 of a surgical saw through the cutting guide slot 182 to engage the patient's tibia 100 and operate the surgical saw to surgically form a planar proximal resected surface of the patient's tibia 100. Once the proximal cut has been made, the surgeon removes the tibial cutting block 180 from the patient's tibia and completes the orthopaedic knee procedure.

As shown in FIG. 9, resection of the tibia 100 may also be performed with the patient's knee positioned in flexion. In such a case, the spacing guides 14, 16 are installed into the patient's knee between the posterior condyle surfaces of the femoral trial component 160 and the articular surfaces 106, 108 of the patient's tibia 100. In particular, the surgeon selects a spacing guide 14, 16 with a paddle 20, 22 having a size (i.e., thickness) which corresponds to the desired extension gaps on each side of the patient's knee (i.e., the medial and lateral sides) and installs it between the respective posterior condyle surfaces 170, 172 of the femoral trial component 160 and the articular surfaces 106, 108 of the patient's tibia 100. For example, if the surgeon previously determined that the medial side of the patient's knee has a desired extension gap of 4 mm, the surgeon selects a medial spacing guide 14 with a size 4 (4 mm thick) paddle 20 and installs it between the medial posterior condyle surface 170 of the femoral trial component 160 and the medial articular surface 106 of the patient's tibia 100. Similarly, for example, if the surgeon previously determined that the lateral side of the patient's knee has a desired extension gap of 2 mm, the surgeon selects a lateral spacing guide 16 with a size 2 (2 mm thick) paddle 22 and installs it between the lateral posterior condyle surface 172 of the femoral trial component 160 and the lateral articular surface 108 of the patient's tibia 100. Once the spacing guides 14, 16 have been installed, the surgeon may then align and pin the tibial cutting block 180 to the patient's tibia 100 by use of the alignment guide 12 in a similar manner to as described above and thereafter perform the resection of the patient's tibia 100.

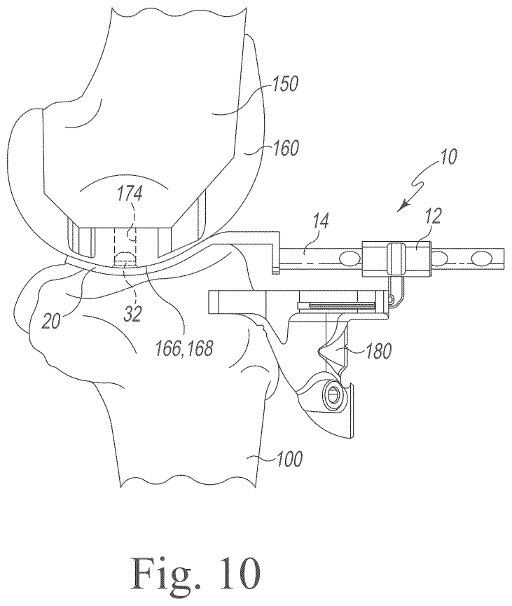

As shown in FIG. 10, in some embodiments, it may be desirable to mechanically couple the paddles 20, 22 to the femoral trial component 160. In such a case, an upwardly-extending post 32 may be formed in the superior alignment surfaces 24, 26 of the paddles 20, 22. The post 32 is sized and shaped to be friction fit into the lug holes 174 formed in the femoral trial component 160 at the dwell points of the component's distal condyle surfaces 166, 168.

As described above, the paddles 20, 22 of the spacing guides 14, 16 are herein shown as being used for gap assessment with the patient's knee positioned in extension or flexion. However, it should be appreciated that the paddles 20, 22 of the spacing guides 14, 16 may be used for gap assessment with the patient's knee positioned in other positions, including positions throughout movement of the patient's knee through a range of motion between extension and flexion.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

1. An orthopaedic surgical instrument assembly for use in the surgical preparation a tibia of a patient during performance of an orthopaedic knee procedure, the orthopaedic surgical instrument assembly comprising:
a femoral trial component configured to be coupled to a surgically-prepared distal end of a femur of the patient, the femoral trial component having (i) an articular side comprising a medial condyle surface and a lateral condyle surface, (ii) a fixation side that is opposite the articular side and configured to engage the surgically-prepared distal end of the femur of the patient, (iii) a medial lug hole positioned at a dwell point of the medial condyle surface and extending from the articular side to the fixation side, and (iv) a lateral lug hole positioned at a dwell point of the lateral condyle surface and extending from the articular side to the fixation side,
a medial spacing guide, the medial spacing guide having (i) a medial paddle that includes a superior alignment surface and an opposite inferior surface, the superior alignment surface being sized and shaped to conform to the medial condyle surface of the femoral trial component, (ii) an elongated shaft extending anteriorly away from the medial paddle, the elongated shaft of the medial spacing guide being secured to the alignment guide, and (iii) an upwardly extending post formed in the superior alignment surface of the medial spacing guide, the upwardly extending post of the medial spacing guide being sized and shaped to be friction fit into the medial lug hole of the femoral trial component, and
a lateral spacing guide, the lateral spacing guide having (i) a lateral paddle that includes a superior alignment surface and an opposite inferior surface, the superior alignment surface being sized and shaped to conform to the lateral condyle surface of the femoral trial component, (ii) an elongated shaft extending anteriorly away from the lateral paddle, the elongated shaft of the medial spacing guide being secured to the alignment guide, and (iii) an upwardly extending post formed in the superior alignment surface of the lateral spacing guide, the upwardly extending post of the lateral spacing guide being sized and shaped to be friction fit into the lateral lug hole of the femoral trial component.

2. The orthopaedic surgical instrument assembly of claim 1, wherein:
the superior alignment surface of the medial spacing guide is sized and shaped to conform to a medial distal condyle surface of the femoral trial component, and
the superior alignment surface of the lateral spacing guide is sized and shaped to conform to a lateral distal condyle surface of the femoral trial component.

3. The orthopaedic surgical instrument assembly of claim 1, wherein:
the superior alignment surface of the medial spacing guide is sized and shaped to conform to a medial posterior condyle surface of the femoral trial component, and
the superior alignment surface of the lateral spacing guide is sized and shaped to conform to a lateral posterior condyle surface of the femoral trial component.

4. The orthopaedic surgical instrument assembly of claim 1, wherein:
the inferior alignment surface of the medial spacing guide is configured to be positioned in contact with a medial articular surface of the patient's tibia, and
the inferior alignment surface of the lateral spacing guide is configured to be positioned in contact with a lateral articular surface of the patient's tibia.

5. The orthopaedic surgical instrument assembly of claim 1, wherein:
the elongated shaft of the medial spacing guide has a longitudinal axis that extends in the anterior/posterior direction, and
the elongated shaft of the lateral spacing guide has a longitudinal axis that extends in the anterior/posterior direction so as to be parallel with the longitudinal axis of the elongated shaft of the medial spacing guide.

6. A method of surgically preparing a tibia of a patient during performance of an orthopaedic knee procedure, the method comprising:
installing a femoral trial component on a surgically-prepared distal end of a femur of the patient,
determining an amount of cartilage loss on an articular surface of the tibia of the patient,
selecting a spacing guide that includes a paddle having a thickness that corresponds to the determined amount of cartilage loss on the articular surface of the tibia of the patient from a plurality of spacing guides each of which has a paddle of differing thicknesses, and
inserting a post of the paddle of the selected spacing guide into a lug hole formed at a dwell point of a condyle surface of the installed femoral trial component so as to install the paddle of the selected spacing guide between the condyle surface of the installed femoral trial component and the articular surface of the tibia of the patient.

7. The method of claim 6, wherein:
determining the amount of cartilage loss comprises determining an amount of cartilage loss on a medial articular surface of the tibia of the patient,
selecting the spacing guide comprises selecting a spacing guide that includes a paddle having a thickness that corresponds to the determined amount of cartilage loss on the medial articular surface of the tibia of the patient from a plurality of spacing guides each of which has a paddle of differing thicknesses, and
inserting the post of the paddle of the selected spacing guide comprises inserting the post of the paddle of the selected spacing guide into a lug hole formed at a dwell point of a medial condyle surface of the installed femoral trial component so as to install the paddle of the selected spacing guide between the medial condyle surface of the installed femoral trial component and the medial articular surface of the tibia of the patient.

8. The method of claim 6, wherein:
determining the amount of cartilage loss comprises determining an amount of cartilage loss on a lateral articular surface of the tibia of the patient,
selecting the spacing guide comprises selecting a spacing guide that includes a paddle having a thickness that corresponds to the determined amount of cartilage loss on the lateral articular surface of the tibia of the patient from a plurality of spacing guides each of which has a paddle of differing thicknesses, and
inserting the post of the paddle of the selected spacing guide comprises inserting the post of the paddle of the selected spacing guide into a lug hole formed at a dwell point of a lateral condyle surface of the installed femoral trial component so as to install the paddle of the selected spacing guide between the lateral condyle surface of the installed femoral trial component and the lateral articular surface of the tibia of the patient.

9. The method of claim 6, wherein:
determining the amount of cartilage loss comprises determining an amount of cartilage loss on both a medial articular surface of the tibia of the patient and a lateral articular surface of the tibia of the patient,
selecting the spacing guide comprises (i) selecting a medial spacing guide that includes a paddle having a thickness that corresponds to the determined amount of cartilage loss on the medial articular surface of the tibia of the patient from a plurality of spacing guides each of which has a paddle of differing thicknesses, and (ii) selecting a lateral spacing guide that includes a paddle having a thickness that corresponds to the determined amount of cartilage loss on the lateral articular surface of the tibia of the patient from a plurality of spacing guides each of which has a paddle of differing thicknesses, and
inserting the post of the paddle of the selected spacing guide comprises (i) inserting a post of the paddle of the selected medial spacing guide into a lug hole formed at a dwell point of a medial condyle surface of the installed femoral trial component so as to install the paddle of the selected medial spacing guide between the medial condyle surface of the installed femoral trial component and the medial articular surface of the tibia of the patient, and (ii) inserting a post of the paddle of the selected lateral spacing guide into a lug hole formed at a dwell point of a lateral condyle surface of the installed femoral trial component so as to install the paddle of the selected lateral spacing guide between the lateral condyle surface of the installed femoral trial component and the lateral articular surface of the tibia of the patient.

10. The method of claim 6, wherein determining the amount of cartilage loss on the articular surface of the tibia of the patient comprises advancing a depth probe into cartilage of the articular surface of the tibia of the patient.

11. A method of surgically preparing a tibia of a knee of a patient during performance of an orthopaedic knee procedure, the method comprising:
installing a femoral trial component on a surgically-prepared distal end of a femur of the patient,
determining, with the patient's knee positioned in extension, (i) a desired medial extension gap indicative of a distance between a medial condyle of the patient's femur and a medial plateau of the patient's tibia, and (ii) a desired lateral extension gap indicative of a distance between a lateral condyle of the patient's femur and a lateral plateau of the patient's tibia,
selecting a medial spacing guide that includes a paddle having a thickness that corresponds to the determined medial extension gap from a plurality of spacing guides each of which has a paddle of differing thicknesses,
selecting a lateral spacing guide that includes a paddle having a thickness that corresponds to the determined lateral extension gap from the plurality of spacing guides each of which has a paddle of differing thicknesses,
inserting (i) a post of the paddle of the selected medial spacing guide into a lug hole formed at a dwell point of a medial condyle surface of the installed femoral trial component, and (ii) a post of the paddle of the selected lateral spacing guide into a lug hole formed at a dwell point of a lateral condyle surface of the installed femoral trial component, and
installing (i) the paddle of the selected medial spacing guide between a medial condyle surface of the installed femoral trial component and the medial plateau of the tibia of the patient, and (ii) the paddle of the selected lateral spacing guide between a lateral condyle surface of the installed femoral trial component and the lateral tibial plateau of the tibia of the patient.

12. The method of claim 11, wherein determining the desired medial extension gap and the desired lateral extension gap comprises operating a ligament balancer to balance the patient's knee.

13. The method of claim 11, wherein determining the desired medial extension gap and the desired lateral extension gap comprises determining an amount of cartilage loss within the patient's knee.

14. The method of claim 11, wherein installing the selected medial and lateral spacing guides comprises installing the selected medial and lateral spacing guides with the patient's knee positioned in extension.

15. The method of claim 11, wherein installing the selected medial and lateral spacing guides comprises installing the selected medial and lateral spacing guides with the patient's knee positioned in flexion.
